(19) United States
(12) Patent Application Publication
(10) Pub. No.: US 2024/0009839 A1
(43) Pub. Date: Jan. 11, 2024

(54) A MEDICAL DEVICE, A METHOD FOR CONTROLLING A DEVICE, A SYSTEM COMPRISING A DEVICE, AND A METHOD OF PRODUCING A DEVICE

(71) Applicant: ARTEDRONE, Paris (FR)

(72) Inventors: Philippe POULETTY, Paris (FR); Maëlle BRUNEAU, Paris (FR); Pierre POUPONNEAU, Paris (FR)

(21) Appl. No.: 17/916,250

(22) PCT Filed: Apr. 1, 2021
(86) PCT No.: PCT/EP2021/058593

§ 371 Date: Sep. 30, 2022

(30) Foreign Application Priority Data
Apr. 1, 2020 (WO) PCT/IB2020/000300

(51) Int. Cl. B25J 9/16; A61B 34/30; A61B 34/00; A61M 37/00

(52) U.S. Cl. CPC B25J 9/1664; A61B 34/30; A61B 34/73; A61B 34/72; A61M 37/00; A61B 2090/3966

(57) ABSTRACT

The present invention relates to a medical device (10), preferably a micro robot for application inside a body, preferably for application inside a human body (2). The medical device (10) includes a body part (11) and a tail part (12). A controlling line (13) is attached to the tail part (12). The controlling line (13) has a tensile strength sufficient to pull back the device from a target location and/or control its velocity and column strength not sufficient to push the medical device (10).

The present invention relates to a medical device and a method for performing a surgical operation in a body. In some nonlimiting examples the medical device relates to a micro robot for application inside a human body.

Minimally invasive procedures, also known as minimally invasive surgeries, are surgical techniques that only require minimal size incisions and therefore require less wound healing time and reduce the risk of trauma in a patient. Specific tools were designed for minimally invasive surgeries such as catheters, fibre optic cables, grippers and pincers on long sticks or miniature video cameras.

A limitation of minimally invasive surgeries is that the surgeon may have to use tools that require a calm hand, which can be exhausting for long operations.

A further development in the field of minimally invasive surgery is robot-assisted surgery or robotic surgery. Hereby robotic systems are used in surgical procedures to assist the surgeon. Multiple robotic arms may perform a minimally invasive surgery while the surgeon handles the robotic arms e.g. with joysticks. However the operation is still invasive to some extent and creates internal and external wounds, which require healing time.

A further development are micro robots that are injected into the human body to perform a diagnosis, a surgery or a treatment. These micro robots can be used for diagnosis or monitoring a disease in real time measuring glucose levels in diabetic patients or for delivering drugs to a targeted location, for example a tumor (Ornes, 2017, PNAS). These micro robots are small devices and have a size ranging from a few millimetres to a few microns. Thus, microrobots are useful to reach areas near small blood vessels or areas after a tortuous vascular network. These targeted areas are challenging to reach by surgery and minimally invasive surgery.

Edd et al. have disclosed a surgical micro-robot that swims inside the human ureter and is proposed to provide a novel method of kidney stone destruction (Proceedings 2003 IEEE). Peyer et al. disclosed a swimming micro robot with artificial bacterial flagella to navigate in fluids of different viscosities (2012 IEEE). Micro robots are unable to carry batteries and motors due to their size. A popular way to guide the micro robots to the target locations is to control a micro robot comprising magnetic materials using external magnetic fields. The Multi-Scale Robotic lab at ETH Zurich disclosed an untethered micro robot with a diameter of 285 μm to perform eye surgeries.

Several insertable medical devices are known in the art and disclosed, for example in US 2013/0282173 A1, US 2008/0058835 A1, U.S. Pat. No. 6,240,312 B1, US 2009/0076536, JP 2002/000556 A, and DE 10 2005 032371 A1.

The small size of these micro robots limits their ability to move against a fluid stream such as the blood stream. Magnetic fields can guide or stop the robot, but might not be strong enough to move the robot quickly inside a blood stream, in particular against a blood stream flowing in an opposite direction to the direction of travel of the microrobot.

The invention seeks to mitigate one or more of the above issues, in particular to provide a medical device, preferably a micro robot that is simple to produce and to use. Some embodiments have the additional advantage of being reliable and enabling a safe recapture.

According to the invention the problem is solved with the characterising features of the independent claims.

The invention relates to a medical device. The medical device may be a micro robot for use in a body vessel. In particular, the medical device or micro robot may be suitable for application inside a human body. The medical device includes a body part and a tail part. A controlling line is attached to the device, preferably to the tail part and may be adapted to pull the medical device back from a first position and/or control its velocity. In one embodiment, the controlling line may have a stiffness not sufficient to move the medical device to a target location.

The first position may in particular be a target site of the medical device.

Preferably, the controlling line is a recapture line intended to brake and/or stop the medical device.

In particular, it is possible to use the controlling line to control the velocity in a fluid stream, for example a blood stream, in particular if the controlling line is attached to the tail part. The controlling line may have a tensile strength sufficient to pull back the medical device and may have a column strength not sufficient to push the medical device against a force created by static or dynamic body fluids. Therefore, the line can be formed sufficiently thin such as to be easily insertable into a body duct.

As used herein the term “line” is intended to cover any construction to fulfil the task of pulling the device while optionally being able to fulfil other non-limiting tasks as well.

The medical device may be adapted to be injected into a body, in particular a human body. The tail part and optionally the body part may have a larger cross-section than the controlling line. The medical device may be retained or pulled back mechanically or manually. Such a controlling line allows pulling the medical device through an opposing fluid stream, for example a blood stream. It also allows controlling the speed of a medical device which is carried by the blood stream, slowing down, or stopping the device within the patient's body, despite the blood stream. This pulling movement can be a slight adjustment of the position or a recapture of the medical device. In particular, the device may comprise a handle for the controlling line.

The controlling line may have a length configured to extend from the medical device to an insertion site of the medical device. One embodiment of the invention relates to a system comprising a port and a medical device wherein the controlling line extends from the tail part to the port.

The controlling line may be a string, in particular a flexible string. Advantageously, the string is bendable. An advantage is that such medical devices can be small in size and only require a small incision, as compared to the known catheter devices.

The medical device can be released into a body vessel, carried through the body vessel by a fluid flow to a target site in a vessel, and recaptured in a simple manner. The device may be repositioned by loosening the controlling line or pulling the controlling line.

The medical device has preferably at least one drive for actively moving the device in a direction and a control member for controlling and preferably changing the movement of the medical device inside the body. The medical device can move within a body fluid flow and/or moving on a tissue.

The drive can be any kind of functionality that moves the medical device. Possible embodiments could be a propeller, wheels, a continuous track (for example a caterpillar track), flagellum, legs, hooks or a magnetic drive for external steering. The control member can move or steer or stop the device by external effects e.g. signals. The control member can adjust the speed or rotation direction of the drive and thereby control the position. The drive may allow the medical device to navigate through sharp turns in the blood vessels.

The medical device has preferably a positioning means to determine the position of the medical device in the body. The positioning means emits a signal, which is received by a receiver.

The receiver then calculates the position of the medical device. This signal could be a radio wave, radioactive tracer, sound wave, Bluetooth, or any other wireless signal. In an alternative embodiment the positioning means could include sensors for measuring different environmental parameters such as temperature, pH, redox potential, salt concentration, viscosity, pressure, electric potential, gas concentration, radioactivity and or metabolic levels. The positioning means sends the measured parameters to the receiver, which calculates the position of the medical device. The measured parameters could also be used to analyse the environment.

The controlling line of the medical device preferably comprises a transmission cable to transmit energy and/or data, in particular light or electric signals from or to the medical device. The transmission cable could include two separate cables, one for delivery of energy and data and one for receiving data. The transmission cable could also be a single cable to transmit energy and data and figure as a controlling line. Additionally or alternatively, the transmission line may be a micro-coaxial cable.

The controlling line may comprise or consist of a biocompatible material. The controlling line preferably comprises or substantially consists of a material selected from a group of materials consisting of a metal, in particular copper, stainless steel, cobalt-chromium-nickel alloys, titanium, titanium alloys, platinum, platinum alloys, Nitinol, nickel-titanium ternary alloys, nickel-free alloys; metal composites but also polymers, carbon fibres, graphene, a fabric, silk, protein fibers, and carbon nanotubes.

Particularly suitable polymers are aramide, in particular one of Kevlar and Twaron, polyamides (in particular Nylon, i.e. PA 6 and PA 66), polytetrafluoroethylene, silicone, polyurethanes, polyvinylchloride (PVC), bioresorbable polymers such as polyglycolic acid (PGA), polydioxanone (PDO), polylactic acid (PLA, in particular one of PLLA and PDLA, and/or their corresponding copolymers such as P(LA-GA)), poly-8-caprolactone and its corresponding copolymers (for example P(LA-CL)). In addition, collagen and chitosan are natural polymers that are equally suited as controlling line materials.

It will be understood that any of the above-listed polymers may be blended, mixed or used as respective co-polymers.

A particularly suited metal is magnesium and magnesium alloys. Magnesium can be biocorrodable and biocompatible. In addition, its corrosion (and thus degradation) rate may be tuned by alloying and/or accelerated by applying a voltage. This property can, for example, be used to release the medical device or a part of the medical device.

These materials have sufficient biocompatibility such that they do not degrade or cause adverse effects such as thrombosis over the time span of a treatment. This ensures that the medical device can be removed at any time, if necessary. Additionally, the materials resist environmental impact in the body, such as different pH or oxidative stress for a certain time frame. Typically, such a time frame is a few hours, but may be anywhere between 1 and 60 min or between 1 and 6 hours. In addition, they have a sufficient longitudinal strength for pulling the medical device. Further, the above materials resist degradation, preferably at least for a few hours or days. Some materials may degrade later (i.e. over a longer period of time that the treatment requires). For example, a slower degradation may be employed if a medical device, or a part of it, is left in the human body after treatment intentionally or as a safety mechanism if a device is lost in a human body.

Preferably, the controlling line has a smaller cross-section, in particular in a plane perpendicular to a longitudinal direction of the controlling line, than the medical device. The cross-section of the line may be less than 50% of the cross section of the medical device.

The medical device preferably comprises a material that is detectable by imaging techniques e.g. by MRI, CT scanner, echography, X-ray or fluoroscopy.

Thereby, the position of the device can be determined at any time during the procedure. If necessary, the positon can also be tracked, in particular in real time. A continuous localization process is beneficial, since the guiding of the medical device can be complicated depending on parameters such as viscosity of the fluid or external pressure from a body fluid stream.

The medical device may be particularly suited for blood vessels, in particular arteries or veins. Other areas of application may be the urethra or the ureter.

The controlling line of the medical device has preferably an outer diameter of 10 to 1000 μm and more preferably of 100-400 μm.

The body part may comprise a magnetic part. This magnetic part is usable to guide the medical device by interaction with an external magnetic field. The magnetic part may be an inner core made of a magnetic material or comprising a magnetic material, magnetic micro- or nanoparticles in a matrix or a coating.

The medical device comprises preferably at least one functional unit such as a clamp, scalpel, drill, hook, stent, legs, caterpillar, propeller, detonator, camera or a sensor or a drug release component.

The functional unit can be attachable to the medical device. The functional unit can be used to move the medical device on a tissue or through a fluid. It can also be used to attach the medical device to a tissue site or to open a passage through a blocked opening or to create a new opening. Alternatively the functional unit can be used to collect data from the body environment.

The proposed device is particularly suitable to remove thrombosis in arteries, fill aneurysms or deliver drugs to a tumor. The detonator could open a thrombus.

The functional unit may be activatable. In some embodiments the functional unit is activated with a magnetic field or electromagnetic waves in certain embodiments. This allows e.g. controlled release of a drug. The functional unit may be activatable by energy, e.g. electrical signals.

The functional unit may be attached or attachable to the medical device and/or the controlling line. In particular, the functional unit may be grafted to the controlling line behind the medical device either directly adjacent to the medical device or at a distance to it.

Similarly, it is also possible to attach two or more medical devices to the same controlling line. Such a plurality of medical devices may be attached in a series (i.e. as a chain of medical devices), or in parallel, or in any other arrangement (circle, tree line, etc.).

The medical device comprises preferably a reservoir to store and release a drug. The reservoir can be used to apply drugs to specific application sites. For example tumor cells can be locally treated with a toxic drug. The medical device is thereby used to transport the toxic drug to the application site and release it there. The controlled release of drugs enables also the possibility of timed drug application. The medical device can be inserted, guided to the site of application and wait until the scheduled release time of the drug. It is also possible to control the delayed release of two different drugs for example an active drug and an enzyme to deactivate the drug.

The medical device comprises preferably a transmitter to send data from the medical device to a receiver, particularly through the controlling line.

The controlling line may be adapted to transmit energy. Thereby, data obtained by a sensor in the medical device may be transmitted.

The device could be adapted to receive energy trough the controlling line and/or send data obtained by sensors in the medical device, in particular in the body part, through the controlling line. In additional or alternative embodiments, the medical device or the controlling line may comprise a wireless transmitter and/or a wireless receiver for sending and/or receiving energy or data.

The medical device has preferably a size of 8-2000 μm, preferably 50-1000 μm and more preferably 200-500 μm. The size may be a length, a diameter, or a longest dimension of the medical device.

The body and/or tail part of the medical device preferably comprise a material such as metal, plastic, glass, mineral, ceramic, carbohydrate, nitinol, carbon, biomaterial, or a biodegradable material.

Preferably, the controlling line is removably attached to the medical device. This enables to detach the medical device from the controlling line. Any mechanism to detach an element from a string-like element known in the art may be employed to this end. For example, the controlling line may be glued to the medical device, wherein the adhesive connection dissolves in blood or another liquid. It would also be conceivable to adapt an adhesive such that is only dissolve in blood above or below a certain temperature.

In particular, the controlling line may be chemically linked to the medical device, wherein the chemical connection could be broken under specific conditions, such as temperature increase, change in pH, electrical stimuli, or similar.

Mechanical means are also conceivable. For example, the controlling line may be attached to the medical device via a hook, a knot, a carabiner and/or a clamp. Additionally or alternatively, the medical device may be at least partially penetrated by the controlling line and anchored either within the medical device or on a surface of the medical device, in particular on a surface that is arranged on an opposite side of the medical device compared to the controlling line. It is also conceivable to use a mechanical interlock, i.e. a first and a second contour that interact with each other such that they connect two elements. It is particularly advantageous to use a mechanical interlock mechanism in combination with an adhesive, because the connection is then provided through cohesive forces in the adhesive as opposed to adhesive forces between the adhesive and the medical device/controlling line.

Similarly, a chemical or physical detachment (chemisorption, physisorption, magnetic and/or electric fields) are also conceivable.

For example, an anchoring point, the medical device or a part of the medical device may at least partially be formed by ferrous material. Applying an electrical current and/or an electrical voltage to the controlling line can cause migration of ferrous ions from the anode to the cathode, which causes dissolution of the ferrous part. Additionally or alternatively, the controlling line may have an insulating portion to protect a part of the controlling line and/or device from corroding and dissolving.

Preferably, the controlling line is selectively detachable from the medical device. In particular, the selective detachment may be triggered by an electrical stimulus, a magnetic part rotation, a physical action, and/or a chemical action.

For example, the controlling line may be adapted to transmit an electrical signal that detaches the controlling line from the medical device. Similarly, this may be done by means of a magnet and/or electromagnet. The medical device may also receive a wireless signal, for example through a wireless signal receiver, that selectively triggers detachment of the medical device from the controlling line.

Additionally or alternatively, the medical device may also detect a property of its surrounding tissue/liquid and release the controlling line automatically. For example, it may detect a temperature, pH, body flow value, inflammation value, or a biomarker and release the controlling line based on that value.

Preferably, the medical device comprises a first and a second portion. In particular, the first portion may be the tail part and the second portion may be the body part. The first portion is attached to the controlling line. The second portion is removably attached to the first portion. The first portion is selectively detachable from the second portion of the medical device, in particular by at least one of an electrical stimulus, magnetic part rotation, physical action, chemical action.

In particular, any mechanism described above as suitable to selectively detach the controlling line from the medical device is also suitable to selectively detach the first from the second portion.

Preferably, the medical device comprises exactly one line formed by the controlling line. The medical device may, in particular, not be attached to any other elements that extend from it, such as cables. If the medical device comprises exactly one controlling line, and said controlling line is selectively detached from the device, the device is then free-floating in its environment.

Preferably, the controlling line is not able to transmit data or energy. It may be made of non-conductive materials, or not be able to conduct electricity along its longitudinal direction for example because of its structure (such as a sandwich structure comprising an insulator). The controlling line may be made of metal, but a connection to the medical device may be unsuitable to transmit electricity, for example because the connection is made of or coated with an insulating material. As such, additionally or alternatively, the device may be unable to receive data or energy through the controlling line.

Preferably, the controlling line can be bent into a curve with a curvature radius of less than 3 mm, preferably 1 mm, even more preferably 700 μm without substantial material stress. The person skilled in the art will understand that the above curvature radii refer to an otherwise straight controlling line (i.e. theoretical stress of 0 Pa at a curvature of 0, i.e. a radius of infinity). In particular, the controlling line may be made of a material and/or structure such that the minimum breaking stress (i.e. the mechanical stress in the controlling line before plastic deformation and/or material failure occurs) is in the range of 0.5-4 MPa. The person skilled in the art understands that it is possible to carry out the invention with a controlling line haver higher breaking stress (i.e. a more robust controlling line). However, higher values may not be required. For the invention to work.

The elastic modulus of the controlling line may be in the range of 0.001 to 200 GPa. Preferably, a controlling line comprising or consisting of a polymer material may have an elastic modulus of 0.001 to 5 GPa. A controlling line comprising or consisting of a metal may have an elastic modulus of 30 to 200 GPa. Of course, materials may be mixed, blended, or used in a combination such as a composite material in order to achieve any elastic modulus. In particular, a composite material of polymers and metals may be used to achieve an elastic modulus anywhere in the range of 0.001 to 200 GPa.

The breaking stress of the controlling line is typically not reached when controlling the medical device. If the controlling line is a NiTi wire, the ultimate tensile strength (UTS) may reach 1300±200 MPa, for polymers wire UTS may be between 30 and 900 MPa.

Preferably, the controlling line comprises or consists of a radio-opaque material such as a barium compound, iodine, tantalum, platinum, bismuth, or a polymeric material.

Preferably, the radiopaque material is arranged as a separate cable associated with and parallel to the controlling line and/or as a coating on the controlling line. This enables a user to directly image the controlling line and to determine its position in a patient's body. Additionally or alternatively, it is also possible to include one or a plurality of radiopaque markers along the controlling line. The radiopaque markers may be arranged at fixed distances or in a random distribution along the controlling line.

Preferably, the controlling line comprises a hydrophilic surface, such as a surface functionalized with PEG (polyethylene glycol) or polyvinylpyrrolidone (PVP) or poly(vinyl alcohol) (PVA) or polytetrafluoroethylene (PTFE), or any combination thereof. Such a surface enables wetting by blood thus enables easier and safer navigation in the blood. In addition, a hydrophilic surface can limit protein adsorption on the controlling line and thus prevent triggering of the immune cascade for instance.

The hydrophilic coating may have a thickness of 50 nm to 10 μm, preferably 100 nm to 500 nm.

The hydrophilic coating can be made by grafting the hydrophilic molecules. The grafting can be done with or without a surface preparation of the controlling surface. The surface preparation can be a chemical etching or mechanical polishing for example. The grafting can be done with a chemical process such as chemical vapor deposition or electro-chemical deposition for example.

The grafting can be done according to a physical process such as physical vapor deposition, layer deposition, spraying or electro-spraying.

The hydrophilic coating may be a surface functionalization.

In an alternative design, the hydrophilic coating can be made with a hydrophilic liner such as PTFE liner.

Preferably, the controlling line comprises a surface with antithrombogeneic properties. For example, it may be coated with a material that does not cause substantial thrombosis. In particular, a surface may comprise at least one of phosphorylcholine, phenox, polyvinylpyrrolidone, and polyacrylamide. Additionally or alternatively, the controlling line may be coated with a drug with an anti-thrombogeneic effect.

Preferably, the controlling line has a surface that is coated with a hydrogel. The hydrogel may be a synthetic hydrogel and/or a natural hydrogel. Preferably, a hydrogel selected from a group comprising elastin-like polypeptides (ELP), polyethylene glycol (PEG), 2-Hydroxyethyl methacrylate (HEMA), polyhydroxymethacrylate (PHEMA), polyvinylpyrrolidone, polymethacrylic acid (PMA) (as well as other methacrylate-based and methacrylic acid-based polymers), agarose, hyaluronic acid, methyl cellulose, elastin, and chitosan is used. Both synthetic hydrogels (ELP, PEG, HEMA, polyvinylpyrrolidone, PMA) as well as natural hydrogels (agarose, hyaluronic acid, methyl cellulose, elastin, chitosan) may be chemically crosslinked and/or physically crosslinked. Other materials that at least partially reduce friction between the controlling line and the vessel walls may also be used.

All types of hydrogels known in the art, in particular homopolymers, copolymers, polymer blends, interpenetrating networks, self-assembled structures, and mixes of polymers may be employed to carry out the invention.

Additionally or alternatively, elastic and/or soft buoys may be attached along the controlling line, allowing for smoother navigation of the controlling line.

Preferably, the controlling line is attached to the medical device by at least one of a knot, a clip, a welded connection, an adhesive connection, a mix of materials, and a chemical bonding.

A mix of material may be provided for forming the controlling line, in particular disposed with a gradient of material composition along a direction of the controlling line. For example, a tail part of the device may comprise a first polymer, while the controlling line comprises a second polymer. The first and the second polymer may be connected via a gradient blend of the first and the second polymer. In particular, the controlling line may also comprise a structure, such as a braided and/or twisted structure, or another multifilament structure. Alternatively, a monofilament may be used. If a multifilament structure is used, all filaments may consist of the same material, or different filaments may be used.

Preferably, the medical device comprises a hollow tube arranged in parallel with respect to the controlling line. The hollow tube is particularly adapted for a suction action, wherein the hollow tube creates a depression causing surrounding fluid and/or tissue to be sucked into the hollow tube. The suction action can be used to help to remove a clot or to stabilize the microrobot on a tissue. Moreover, the hollow tube could be used to inflate a balloon.

Preferably, the medical device further comprises a trigger wire. Particularly preferably, the trigger wire may be associated with and arranged in parallel with respect to the longitudinal direction of the controlling line. For example, it may be arranged inside of the controlling line. Alternatively, it may be arranged next to the controlling line as a separate element, but preferably associated with the controlling line. The trigger wire is adapted to trigger a function of the device.

The trigger wire may, in particular, transmit a mechanical or an electrical signal and may in particular trigger the function of releasing a drug or selectively detaching the medical device from the controlling line.

The trigger wire may have a diameter between 10 μm to 150 μm, preferably between 20 μm to 70 μm. The trigger wire can have a cylindrical shape or strip shape. The trigger wire can be made of a polymer such as PET, or a metal such as nitinol or stainless steel. In one configuration, the trigger wire can transmit a mechanical force to retract an element of the head.

The invention further provides a method for performing a surgical operation in a body, preferably a human body. In a first step a medical device is inserted into a body. The medical device is then navigated to a place of interaction, without pushing a controlling line. In particular, the medical device is inserted upstream of a target site. A fluid stream may carry the medical device to the target site. The medical device may be positioned and/or guided along a trajectory by loosening or pulling the controlling line. The medical device may perform one or several actions at one or several places. The medical device is removed from the body by pulling on the controlling line.

The invention further provides a system for controlling a medical device. The system comprises a medical device, preferably a medical device as described before and a magnetic field generator. The medical device is then guided by a magnetic field generated by the magnetic field generator.

The external magnetic field generator creates a magnetic field with a gradient of 0.1 to 20 T/m, preferably of 0.2-1 T/m. Once the medical device is inserted into the body, the magnetic field can be used to guide the medical device to the application site. Therefore the medical device is moved or stopped or steered by the magnetic field, in particular while floating in a body fluid stream. During the entire time the medical device remains attached to the controlling line.

In further embodiments, the medical device may have a magnetic anisotropy. Thereby, the medical device can be oriented by the magnetic field.

The invention further refers to a medical device, preferably a micro robot for application inside a body, preferably a human body.

Preferably, the system further comprises a control adapted to control the velocity of the medical device, preferably continuously. The control may in particular control the velocity of the medical device by controlling the velocity of a controlling line attached to the medical device. The control may in particular comprise a reel to reel-in and/or release the controlling line.

Preferably, the system further comprises a coupling element adapted to be coupled to the controlling line in order to connect the coupling element to the device to control its velocity, in particular continuously.

The system is preferably adapted to pull in and/or release the controlling line, preferably continuously, at a controlled velocity.

The velocity of the controlling line and thus of the medical device may be controlled according to a pre-determined velocity function or based on a position of the medical device.

Particularly preferably, the velocity and position of the controlling line are adjusted using a linear motor and/or a spindle/reel mechanism.

Preferably, the system comprises a mechanism to control the position of the microrobot, preferably by controlling the release of the controlling line. It may, in particular, comprise a sensor that measures a pull-in/release distance. It may also comprise a servomotor that automatically determines a degree of release of the controlling line.

The continuous release of the controlling line or the stop of the release can be controlled as a function of the position of the medical device, in particular with respect to a targeted trajectory.

The invention is further directed to a method of controlling a device in a fluid stream. The device is preferably a microrobot, and even more preferably a device as described herein above. The fluid stream is preferably blood in a blood vessel. The device comprises a controlling line attached to the device. The velocity of the device is controlled via the controlling line.

Preferably, the velocity is reduced when the device approaches a bifurcation. This enables a more precise navigation along a desired path and thus a safer navigation.

Preferably, a control is provided which automatically controls the velocity of the device, preferably by applying a force to the controlling line.

Preferably, the control automatically detects bifurcations. Such a detection may be based on external imaging, such as ultrasound imaging, MRI, tomography, X-rays, or other known methods. It may, additionally or alternatively, be based on measurement data acquired by the medical device. For example, the medical device may detect flow properties of the fluid which indicate the presence of a bifurcation.

Additionally or alternatively, the medical device may be guided by the control along a pre-planned trajectory initially, for example into a vascular network up to a targeted area.

The invention is further directed to a medical device. Preferably, the medical device is any medical device as described herein. The medical device comprises a magnetic head portion which is attached or attachable to a controlling line. The controlling line is preferably attached or attachable via a first adhesive component. Particularly preferably, the first adhesive component is a cyanoacrylate component or an epoxy glue which are known to the person skilled in the art and commercially available. It will be understood that the first adhesive component may be a medical-grade adhesive. The medical device further comprises a protective layer. The protective layer may comprise, preferably consist of, a second adhesive component. Particularly preferably, the second adhesive component comprises or consists of a resin. The resin is stable in an aqueous environment. The resin may be an epoxy resin. Epoxy resins may provide particularly advantageous water resistance.

Alternatively, the protective layer may comprise or consist of a reticulated polymer. For example, a device comprising a magnetic head attached to a controlling line may be dipped into a polymer solution which is subsequently cured.

The protective coating is configured such as to provide a fluid seal at least of the area of the magnetic head portion which is attached or attachable to the connecting line. In a preferred embodiment, the protective layer is configured to provide a fluid seal of the entire magnetic head portion.

The protective coating can exhibit hydrophilic properties. In one configuration, the protective shell can comprise or consist of a hydrophilic material such as PEG. In one configuration, the protective shell is coated with a hydrophilic material such as PVP or PTFE.

Preferably, the attachment area is arranged at a south pole of the magnetic head portion. Alternatively, the attachment area may also be arranged at a north pole, or in between the south pole and the north pole.

The fluid seal of the magnetic head portion may be entirely or only partially formed by the protective layer. For example, if a controlling line is attached to a round magnetic head portion, the protective layer may not form a closed capsule due to an opening for the controlling line. It will be understood that such an arrangement may still provide a fluid seal of the magnetic head portion.

A protective layer is particularly advantageous as it may provide safer attachment of the controlling line to a magnetic head portion, reduces corrosion effects on the magnetic head portion, in particular in fluids such as blood, and may further provide a further attachment mechanism for therapeutic tools.

It is particularly preferred to provide a protective coating if the medical device comprises a controlling line which is attached by means of a least one of a knot, a clip, a welded connection, an adhesive connection, a mix of materials, and a chemical bonding.

A protective coating may thus reduce or prevent corrosion in particular around an area where the controlling line is attached or attachable. This can be in particular at an interface between an adhesive and the magnetic head portion, if the controlling line is attached to the magnetic head portion via said adhesive.

Thereby a more secure and more stable attachment of the controlling line to the magnetic head portion can be provided.

In particular, it is possible that the breaking force between the magnetic head part and the controlling line is increased as a result of the protective layer. Preferably, the breaking force is at least 1 N, particularly preferably at least 7 N.

Preferably, the protective layer is provided such that the entire magnetic head portion is sealed.

To this end, the protective layer may form a closed capsule. An attachment area, for example formed by a cyanoacrylate adhesive, may be formed on the protective layer surface.

Alternatively, the protective layer may form a seal by cooperating with another element, in particular the first adhesive component, the controlling line, and/or another attachment mechanism between the magnetic head part and the controlling line. It will be understood that the protective layer may form a fluid-tight seal between with these elements as well.

In yet an alternative embodiment, the protective layer is only formed partially around the magnetic head part. As such, the fluid-tight seal may only be provided at an interface between the attachment area and the magnetic head part. For example, the protective layer may be formed as a spherical cap or a spherical segment at least partially covering the attachment area on the magnetic head part.

Preferably, the magnetic head part comprises or consists of a neodymium (Nd—Fe—B) magnet. The magnetic head part may preferably be substantially spherical with a diameter of 0.2-2 mm, preferably 0.7-1.3 mm, particularly preferably 1 mm. The magnetic head part preferably has a residual induction (Br) of 0.52.0 T, particularly preferably 1.0-1.5 T.

Additionally or alternatively, the magnetic part can be made of:
hard ferromagnetic materials such as FePt alloys, Nd—Fe—B alloys, SrO₆Fe₂O₃ alloy,
soft ferromagnetic alloy such as Fe alloys (stainless steel AISI 420C, Fe coated with a protective shell made of graphite for example), Ni alloys, Co alloys, or any combination of these magnetic elements.
ferrimagnetic materials such as iron oxide (Fe₃O₄ or Fe₂O₃) for example.

The magnetic head part may comprise magnetic particles in a polymer matrix. Additionally or alternatively, the magnetic head part may comprise a hollow tube. The magnetic head part may be configured as a Janus particle. The magnetic head part may be configured as a core particle, which may be magnetic or nonmagnetic, with a magnetic shell or coating.

The magnetic head part may further comprise a coating with tracking elements. Tracking elements may, for example, be radio-opaque elements for tracking of a position of the medical device and/or magnetic head part, such as barium compounds, iodine, tantalum, platinum, and/or bismuth. The radio-opaque coating can be made with strips, rings or powder. For example, platinum strips (which may have a width of 100 μm and/or a thickness of 50 μm) can be set up on the surface of the magnetic head.

Alternatively, barium powder can be mixed with a polymer such as epoxy and applied on the surface of the magnetic head. The thickness of coating can be between 1 μm to 70 μm, preferably 5 μm to 15 μm. Particles of radio-opaque powder can have a diameter between 20 nm to 3 μm, preferentially 50 nm to 100 nm.

Additionally or alternatively, tracking elements may also be included in the controlling line.

Thus, it is possible to track and detect a location and/or velocity of the controlling line.

Tracking elements may be grafted, impregnated and/or coated on to the controlling line and/or the magnetic head part.

Additionally or alternatively, the magnetic head part may comprise or consist of hard ferromagnetic materials, soft ferromagnetic materials, ferromagnetic materials, and/or superparamagnetic materials. It is conceivable to combine any of the abovementioned materials in structures. For example, the magnetic head part may comprise a core comprising a hard ferromagnetic material and a shell comprising a soft ferromagnetic materials. Such structures may be advantageous and reduce permanent magnetic aggregation of medical device, in particular if no magnetic field is applied.

The magnetic head portion including the protective layer may have a diameter of 0.2-2 mm, preferably 0.7-1.5 mm, particularly preferably 1.0-1.2 mm.

Preferably, a controlling line is attached or attachable to the magnetic head part via a cyanoacrylate glue.

The controlling line may comprise or consist of multifilament Nylon. The controlling line may have a diameter of 50-500 μm, preferably 100-350 μm, particularly preferably about 200 μm.

The controlling line may have a Young's modulus of 1-50 GPa, preferably 1-20 GPa, particularly preferably 1-3 GPa.

The controlling line may have a bending stiffness of 0.01-1 N·mm², preferably 0.05-0.5 N·mm².

The controlling line may have a rupture stress of 0.1-5 GPa, preferably 0.1-1 GPa, particularly preferably 0.3-0.7 GPa.

The controlling line may have a crosssectional area in a plane perpendicular to a longitudinal axis of 0.001-0.1 mm², preferably 0.004-0.1 mm², particularly preferably 0.01-0.05 mm².

In particular, the controlling line may be adapted, in particular by choice of at least one of material, diameter, cross-section in a plane perpendicular to a longitudinal axis, to have, depending on the diameter, a breaking force in the range of 1 to 20 N, preferably 8 to 12 N.

The invention is further directed to a method of producing a medical device, in particular a medical device as described herein. The method comprises the steps of providing a magnetic head portion with an attachment area attached or attachable to a controlling line. The attachment area may be a first adhesive component, for example a cyanoacrylate. Alternatively, any other attachment mechanism may be used, in particular attachment mechanisms as described herein. The method further comprises a step of providing a protective layer which at least partially covers and/or forms an interfacial area between the magnetic head portion and the attachment area. The protective layer preferably comprises a second adhesive component, in particular a resin.

Preferably, the protective layer is provided as a continuous layer over a surface of the magnetic head part. The protective layer may cover a central portion and/or a proximal portion of the magnetic head part.

Preferably, the protective layer is arranged on a radially outward position with respect to the magnetic head part and the attachment area. However, it is conceivable to arrange the attachment area on the protective layer, i.e. on the outside of the protective layer with respect to the magnetic head part.

Non-limiting embodiments of the invention are described, by way of example only, with respect to the accompanying drawings, in which:

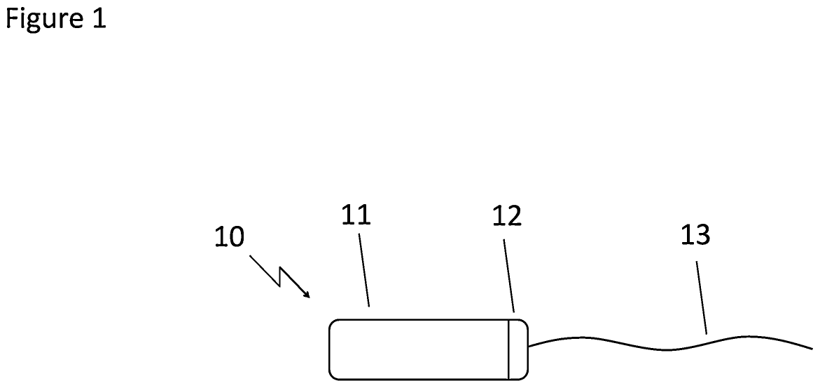

FIG. 1: Schematic view of a medical device.

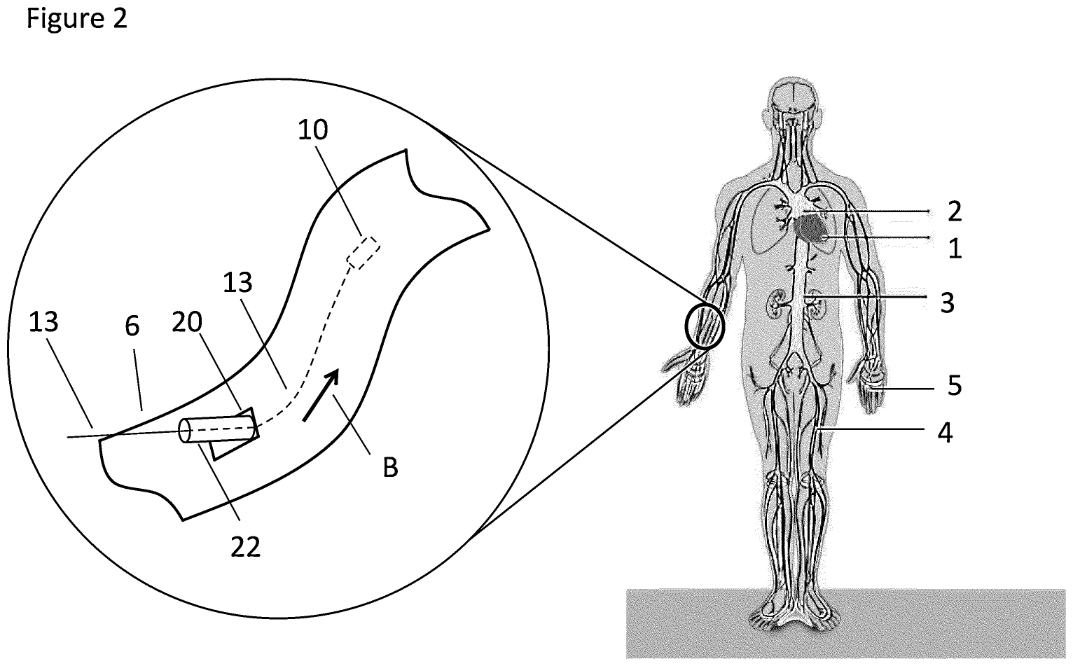

FIG. 2: Schematic view of an insertion site of a human body for the medical device.

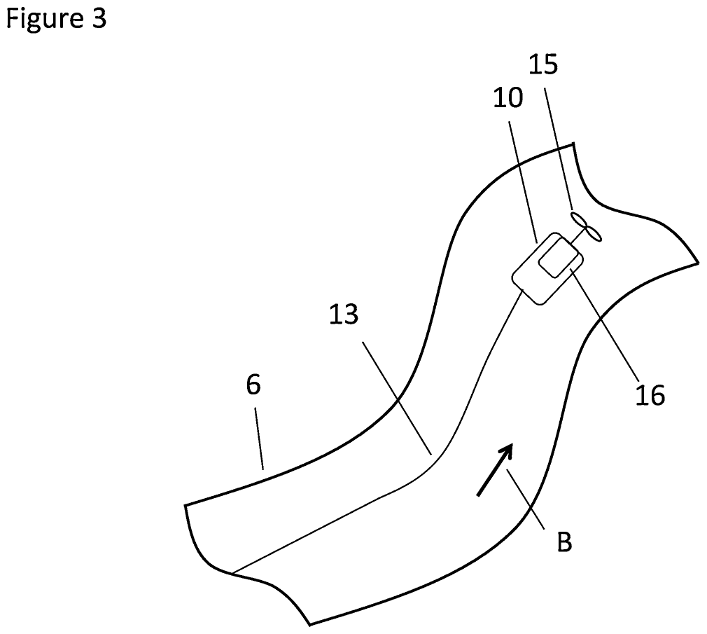

FIG. 3: Schematic view of the medical device with a drive and a control member.

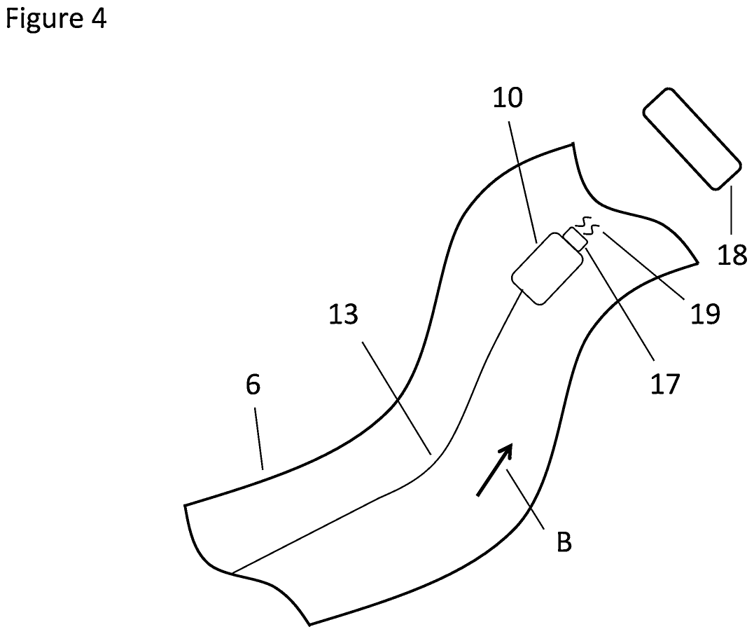

FIG. 4: Schematic view of the medical device with a positioning means.

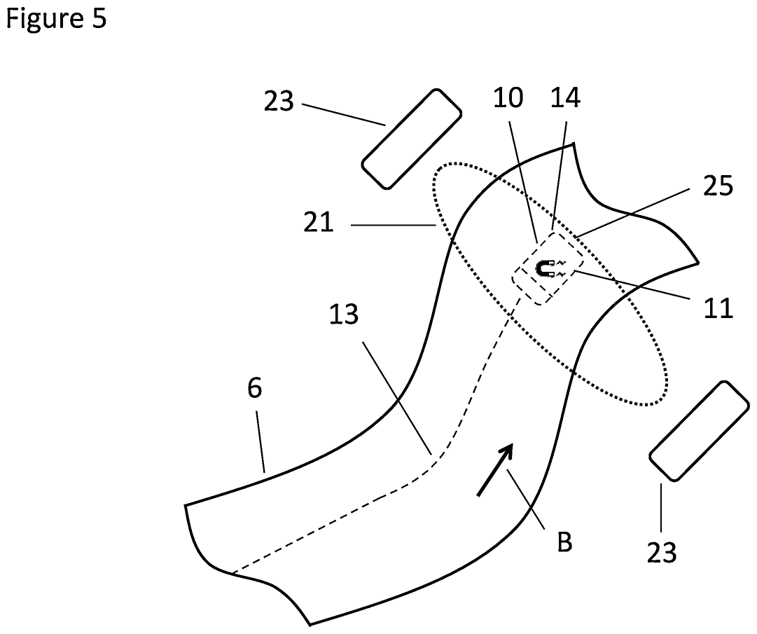

FIG. 5: Schematic view of pulling the medical device with a magnetic field.

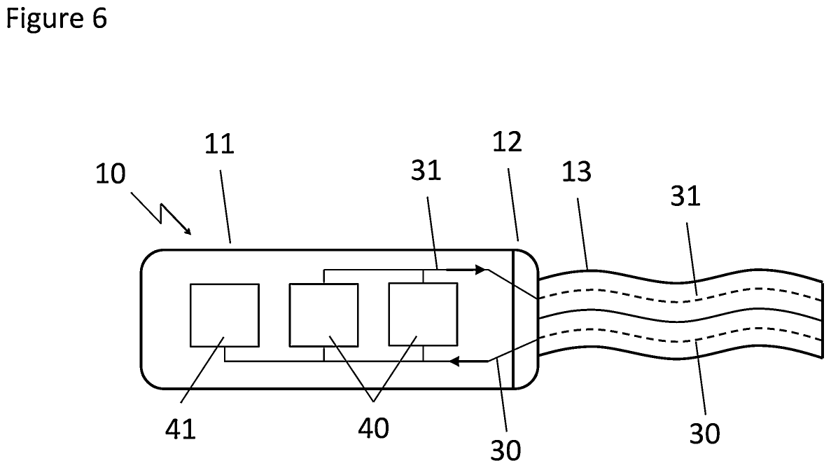

FIG. 6: Schematic view of data and energy transmission through a controlling line of the medical device.

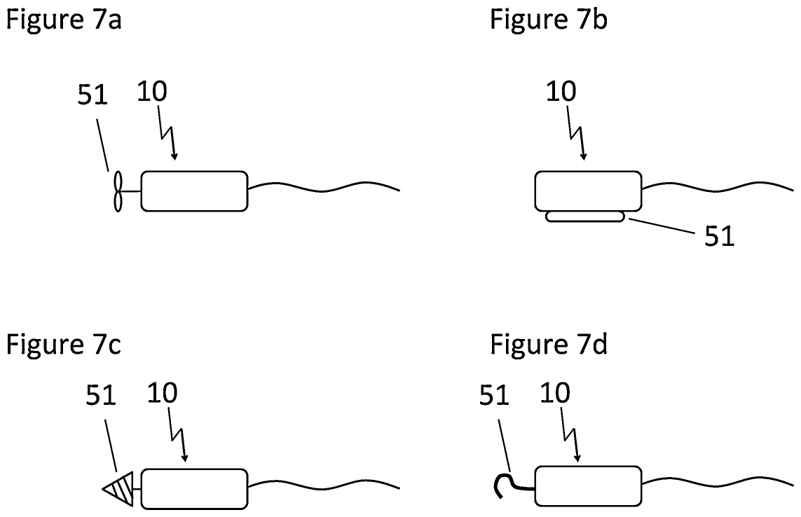

FIGS. 7a-d: Schematic view of functional units attached to the medical device.

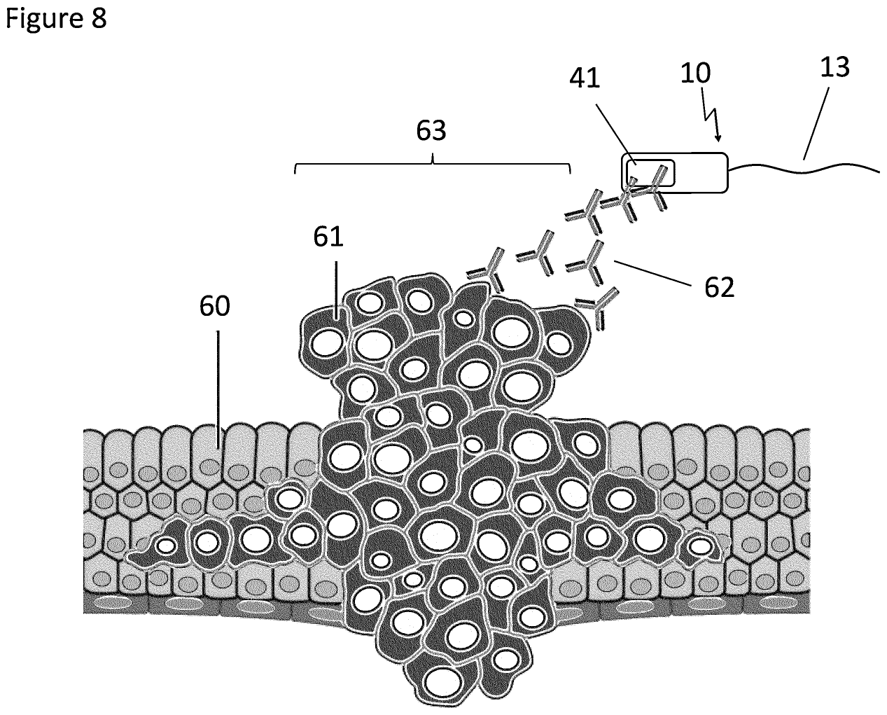

FIG. 8: Schematic view of a tumor and antibodies delivered to the tumor by the medical device.

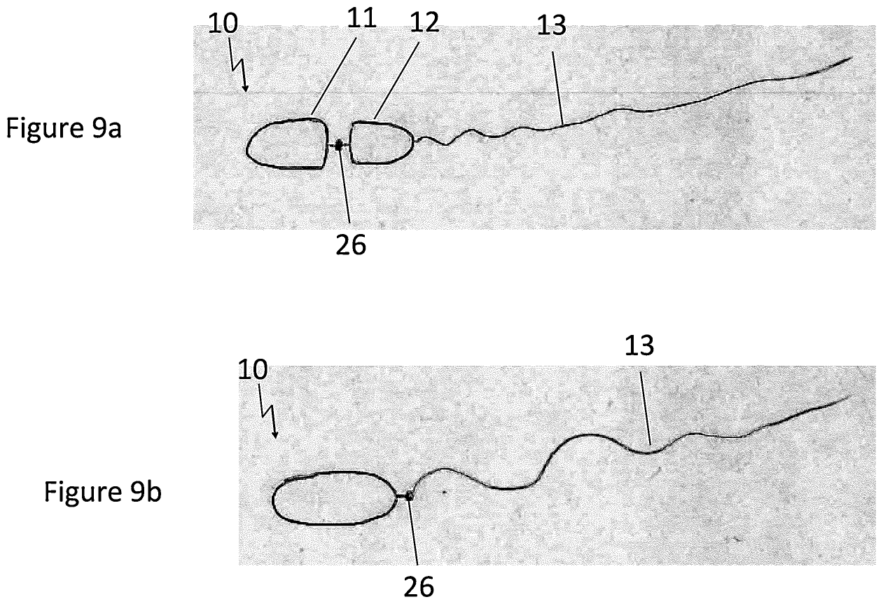

FIG. 9a-9b: different embodiments of a medical device that is releasably attached to a controlling line.

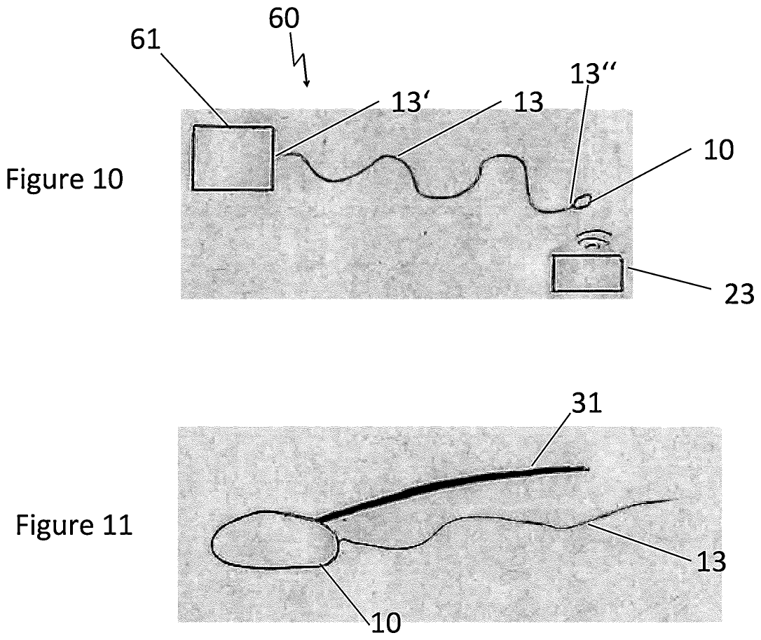

FIG. 10: a schematic view of a system according to the invention.

FIG. 11: an alternative embodiment of a medical device according to the invention.

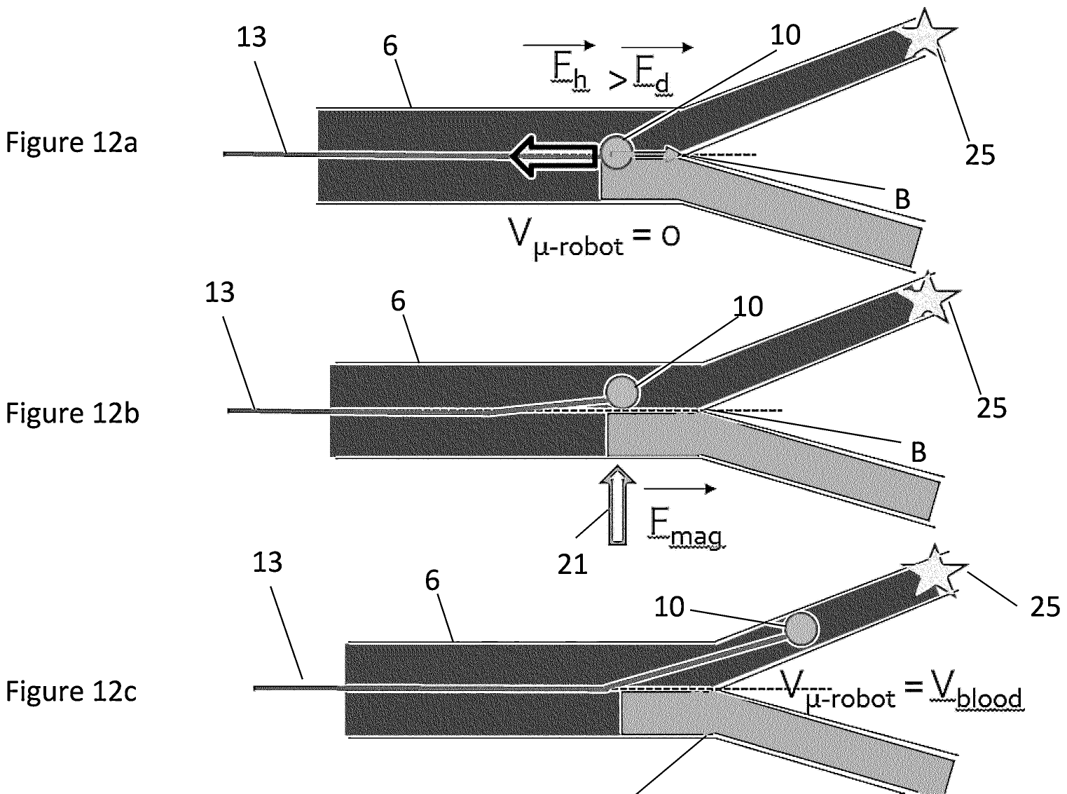

FIGS. 12a-12b: schematically a method according to the invention.

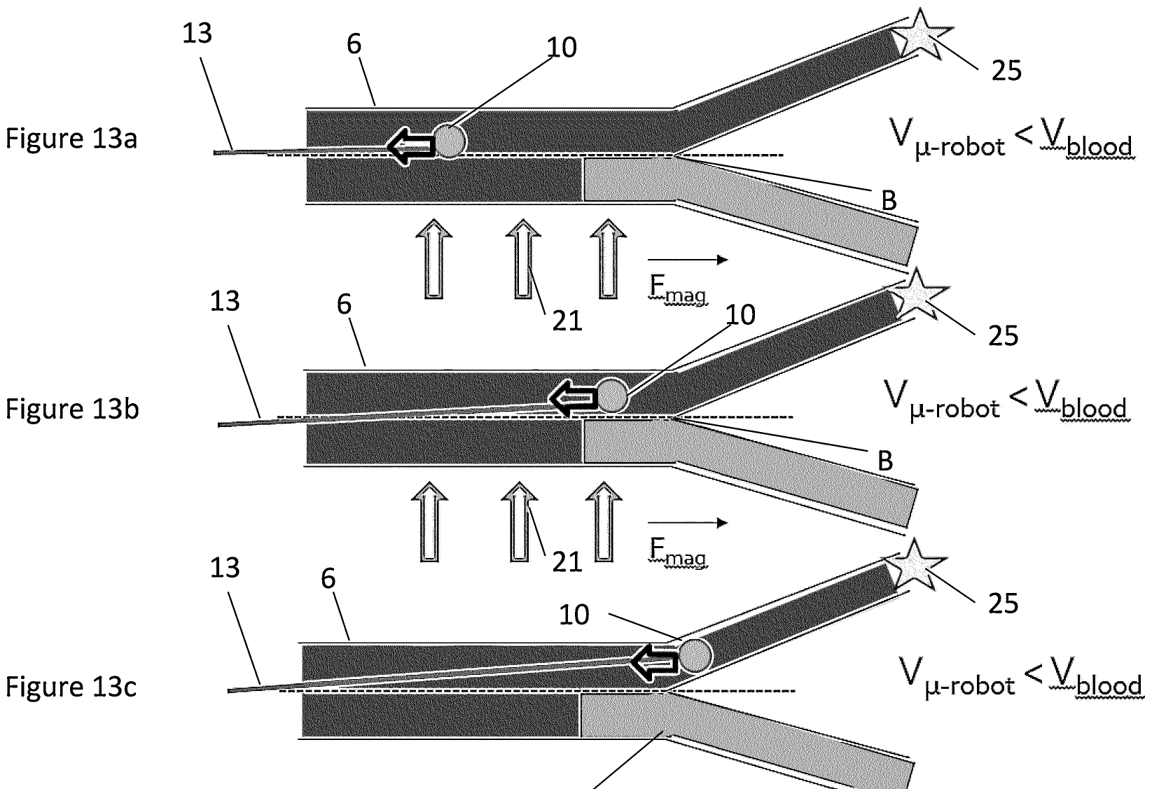

FIGS. 13a-13b: schematically an alternative method according to the invention.

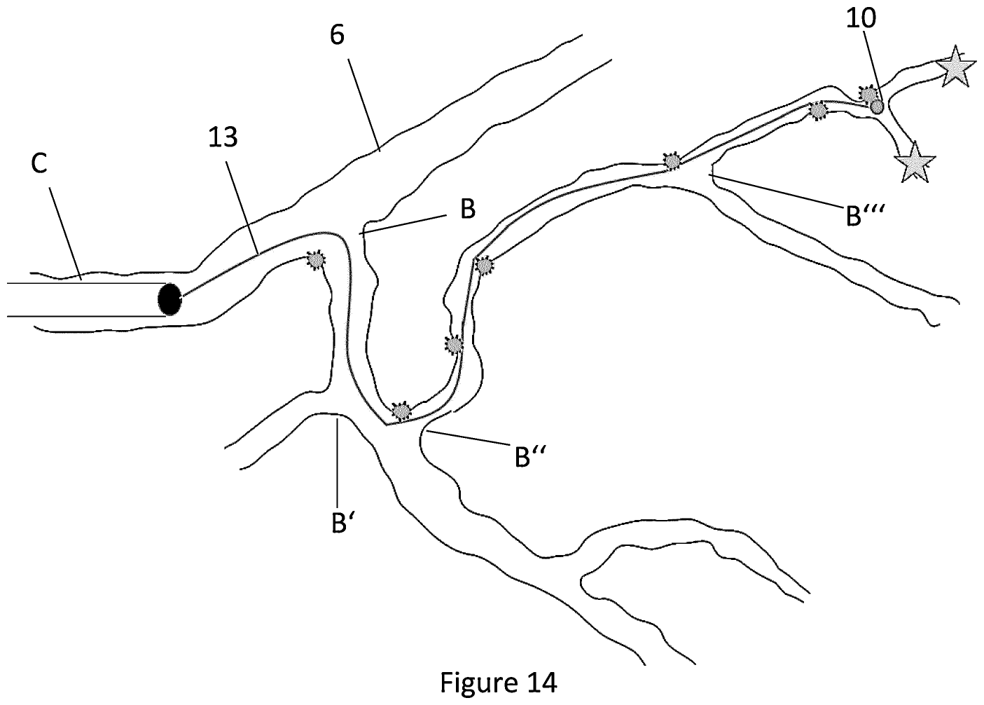

FIG. 14: schematically a medical device inside a vessel filled with blood.

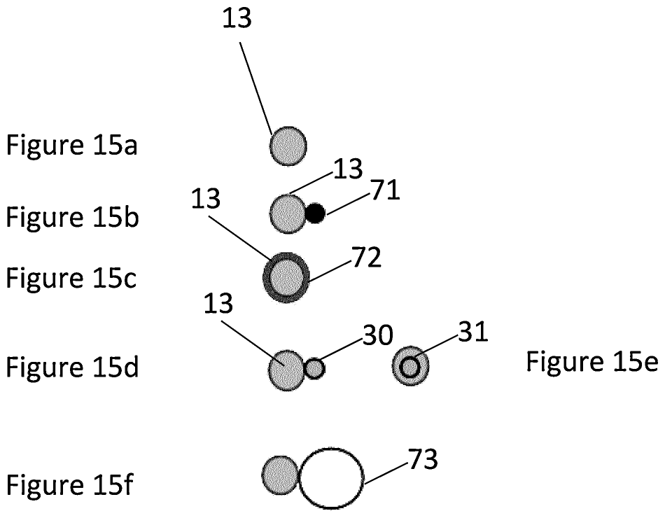

FIGS. 15a-15f: different embodiments of a controlling line with an associated element in a cross-sectional view.

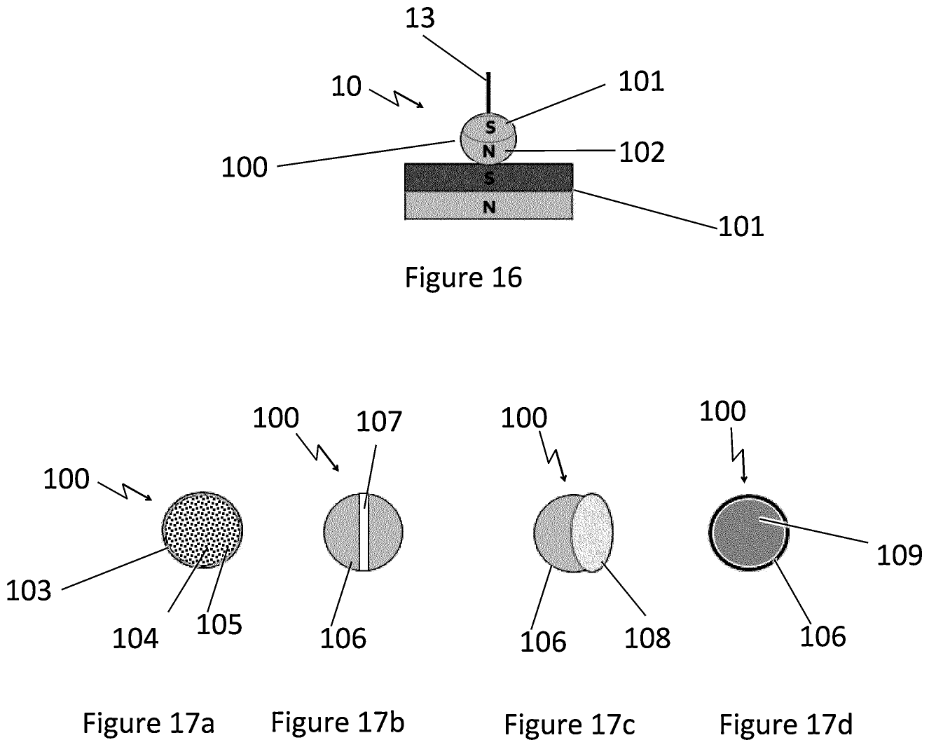

FIG. 16: schematically a method step of producing a medical device.

FIG. 17a-17d: schematically different embodiments of magnetic head parts.

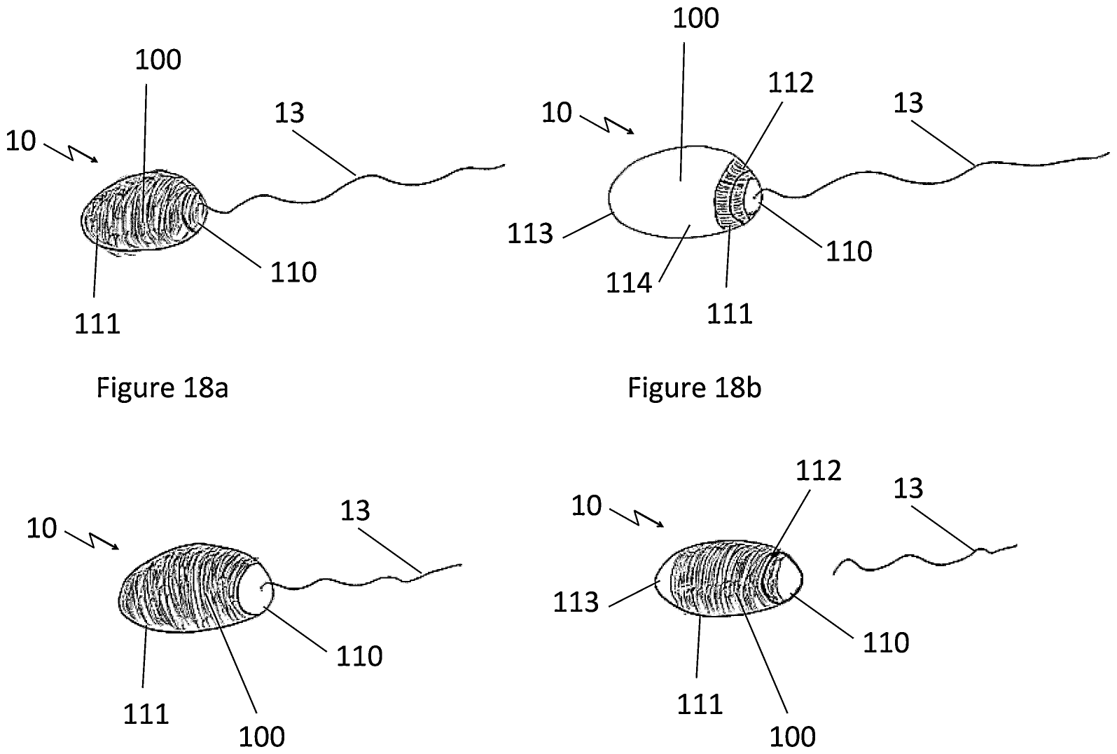

FIG. 18a-18d: schematically different embodiments of medical devices with a protective layer.

FIG. 1 shows a schematic view of a medical device 10 comprising a body part 11 and a tail part 12. A controlling line 13 is attached to the body part 12. The controlling line 13 is used to pull the medical device 10.

FIG. 2 shows a schematic view of an insertion site 20 of a human body 2 for the medical device 10. The heart 1 is connected to a bloodstream. The blood stream comprises different types of blood vessels 6 such as aorta 3, veins 4 and capillaries 5. The medical device 10 is inserted into the blood vessel 6 at the insertion site 20. Therefore the blood vessel 6 is perforated by a catheter 22 at the insertion site 20. The medical device 10 is inserted into a blood stream B. The blood stream B is carrying the medical device 10 through the blood vessel until the medical device reaches a site of interaction 25 (FIG. 5). At any time the medical device 10 is connected to the controlling line 13 and can be pulled back to the site of insertion 20.

FIG. 3 shows the medical device 10 with the controlling line 13 in a blood vessel 6. The medical device 10 has a drive 15 and a control member 16, to control the drive. The drive 15 actively moves the medical device 10 in a direction. The control member 16 modifies the action of the drive 15. The control member 16 can invert the rotation direction of the drive 15 or adjust its speed.

FIG. 4 shows the medical device 10 with the controlling line 13 in a blood vessel 6. The medical device 10 has a positioning means 17. The positioning means 17 emits a signal 19, which is received by a receiver 18. Based on the signal 19, the receiver 18 calculates the position of the medical device 10.

FIG. 5 shows a schematic view of the blood vessel 6 with the medical device 10. The medical device 10 is transported by the blood stream B and attached to the controlling line 13. A magnetic field generator 23 is generating a magnetic field 21 at the application site 25. The body part 11 of the medical device has a magnetic part 14, which is attracted by the magnetic field 21. At the application site 25 the medical device 10 stays in place, held by the magnetic field 21 against force of the blood stream B. After performing any kind of action the magnetic field generator 23 is switched off and the magnetic field 21 collapses. The medical device is removed against the force of the blood stream B by pulling at the controlling line 13.

FIG. 6 shows a schematic view of the medical device 10. The controlling line 13 comprises an energy transmission cable 30 and a data transmission cable 31. The energy transmission cable transmits energy to sensors 40 and a compartment 41. The sensors send data through the data transmission cable 31. As an alternative the energy transmission cable 30 and the data transmission cable 31 can be integrated into the same cable. This cable is used to transport energy to and data to and from the medical device through the controlling line 13.

FIG. 7a-d shows a schematic view of the medical device 10 with attachable functional units 51. In FIG. 7a the functional unit 51 is a propeller to move the medical device 10 in a forward or reverse direction along a longitudinal axis through the device. FIG. 7b shows a medical device 10 where the functional unit 51 is a caterpillar. The caterpillar is used to move the medical device 10 onto a tissue site. In FIG. 7c the functional unit 51 of the medical device 10 is a drill. The drill can be used to perforate a tissue and create an opening to move across physical barriers. In FIG. 7d the functional unit 51 of the medical device 10 is a hook. The hook can be used to hold the medical device 10 in place or to drag an object or material, when the medical device 10 is recaptured.

FIG. 8 shows a schematic view of a tumor site 63. The tumor cells 61 have a bigger size and a faster replication cycle than the normal cells 60. The medical device 10 is guided to the tumor site and carries tumor specific antibodies 62 in the compartment 41. At the tumor site 63 the medical device 10 releases the tumor specific antibodies 62. The antibodies bind to the tumor cells and induce an immunotherapeutic process. After releasing the antibodies 62 the medical device 10 is removed from the tumor site 63 by pulling on the controlling line 13.

FIG. 9a shows another embodiment of a medical device 10 according to the invention. The medical device 10 comprises a tail part 12 and a body part 11 which are configured as separate elements. The body part 11 and the tail part 12 are connected via a connection mechanism 26. The robot is attached to a single controlling line 13 that is adapted to control the robot's velocity in a fluid stream. The connection mechanism 26 is selectively deactiveatable such as to detach the body part 11 from the tail part 12 by applying an electrical current. The connection mechanism 26 comprises a ferrous material that disintegrates when an electrical current flows through it due to electrolysis. The connection mechanism 26 therefore releases the body part 11 of the medical device 10.

FIG. 9b shows an alternative embodiment where a selectively detachable connection mechanism 26 directly connects the controlling line 13 and the medical device 10. The medical device 10 is thus releasable from the controlling line 13 through an electrical detachment similar to the ones described above. The connection mechanism 26 comprises a noble metal part, which comprises a noble metal such as a platinum alloy, that is attached to a ferrous part. By applying an electrical current, the ferrous part acts as an anode and the ferrous ions dissolve in the surrounding liquid and thus disintegrate the ferrous part such as to release the medical device. Additionally or alternatively, the connection mechanism 26 could be degraded by an increase in temperature induced by any known method such as a localized heating element or ultrasound.

FIG. 10 schematically shows a system 60 according to the invention. The system 60 comprises a control 61 that is connected to a first end 13′ of the controlling line 13. A second end 13″ of the controlling line 13 is attached to the medical device 10.

A magnetic field generator 23 is presently included in the system 60 in order to guide or steer the medical device 10 in a fluid stream (not shown).

FIG. 11 shows an alternative embodiment of a medical device 10. The medical device 10 comprises a controlling line 13 for velocity control. The medical device 10 is additionally connected to a transmission cable 31 that transmits data to and from the medical device 10 from and to an external computer (not shown). It would also be possible to transmit electrical energy to the medical device 10 using the cable 31.

FIG. 12a schematically shows a first step of a method according to the invention. A microrobot 10 floats in a vessel 6 in the vicinity of a bifurcation B. According to a treatment plan, the microrobot 10 shall be directed to a target site 25 and thus needs to be steered in the correct direction at the bifurcation B. Thus, the microrobot 10 is slowed down by the controlling line 13 until it comes to a stop at a position upstream of the bifurcation B. The microrobot 10 is now at a fixed position along the streaming direction of the blood, but the microrobot 6 may still perform some limited movements as the controlling line is typically a flexible element.

FIG. 12b shows that the microrobot 10 is pushed towards a target side of the bifurcation B leading to the target site 25. Once the microrobot 10 is positioned, the controlling line 13 may again be released at a controlled velocity such that the microrobot is carried on by the blood again.

FIG. 12c shows the robot moving in the direction of the target site 25 in the blood stream and with substantially the same velocity as the blood flow. When the target site is reached, the robot may be stopped by holding the controlling line.

FIGS. 13a-13b show an alternative method to control a medical device 10 in a vessel 6. The method is similar to the one schematically shown in FIGS. 12a-12c, but differs in that the microrobot 10 is never completely stopped.

FIG. 13a thus shows a microrobot 10 attached to a controlling line in a vessel 6. As the microrobot 10 approaches a bifurcation B, the microrobot 10 is slowed down via the controlling line 13.

FIG. 13b shows how in parallel to the slowdown, a magnetic field 21 is employed to steer the microrobot 10 in a direction of a target site 25.

FIG. 13c shows the microrobot 10 floating again in the blood vessel.

FIG. 14 shows schematically a microrobot 10 in a vessel system 6 with several bifurcations B, B′, B″, B′″. A catheter C is employed to bring the microrobot 10 to a vessel system 6 to be treated. The microrobot's 10 velocity is controlled by controlled release or pull on a controlling line 13 that is attached to the microrobot 10, in particular in the vicinity of the bifurcations B, B′, B″, B′″. The controlling line 13 is made of silk and coated with a hydrogel. For this reason, it is mechanically flexible and can bend to adapt to the vessel system 6. The hydrogel surface additionally reduces the thrombogeneicity of the controlling line 13 and reduces friction on the vessel walls.

FIG. 15a shows a controlling line 13 made of a single material, presently Kevlar, in a cross-sectional view.

FIG. 15b shows a controlling line 13 with a radiopaque line 71 arranged in parallel to a longitudinal direction of the controlling line 13. The radiopaque line 71 consists of a composite of a biocompatible polymer and barium sulphate. It is therefore visible in X-ray imaging. Additionally or alternatively, platinum or gold rings could be associated and connected with the controlling line.

FIG. 15c shows a controlling line 13 with an anti-thrombogeneic hydrogel coating 72 on the surface of the controlling line. Presently, the hydrogel is based on PEG. The hydrogel could however include any material selected from a group of ELPs, HEMA, PHEMA, polyvinylpyrrolidone, PMA (or other methacrylate/methacrylic acid-based polymers), agarose, hyaluronic acid, methyl cellulose, elastin, and chitosan.

FIG. 15d shows a controlling line 13 with a transmission cable 30 for energy transmission configured as a separate element arranged in parallel to the longitudinal direction of the controlling line 13. The transmission line consists of gold and can transmit electrical energy. Alternatively, the transmission line could also consist of platinum, or any conductive metal (such as copper) coated with at least one of gold and platinum. The transmission line could also, additionally or alternatively, be used to transmit data.

FIG. 15e shows an alternative embodiment of a controlling line 13, wherein a transmission cable 31 for data transmission is arranged inside the controlling line 13. Additionally or alternatively, the transmission line 13 may also transmit energy.

FIG. 15f shows an alternative embodiment of a controlling line 13, wherein a hollow tube 73 is arranged in parallel and outside the controlling line 13. The hollow tube is adapted for suction action in order to take tissue samples or to remove fluid and/or cells from the target area.

FIG. 16 shows schematically a method step of producing a medical device. A magnetic head part 100 with a south pole 101 and a north pole 102 is brought in operable contact with a magnet 101 such as to orient the magnetic head 100 part with respect to a magnetic field of the magnet 101. Thus, a controlling line 13 can be selectively attached to the magnetic head part 100 on its south pole 101. Alternatively, the controlling line 13 may be attached to the north pole 102 or any other location of the magnetic head part 100. Usage of a magnet 101 facilitates the attachment because the orientation of the magnetic head part 100 with respect to its magnetic properties may be known.

FIG. 17a shows an embodiment of a magnetic head part 100. The magnetic head part 100 comprises a plurality of magnetic particles 104 arranged within a polymer matrix 105.

One possible method to produce such an embodiment is described in the following. Demagnetized hard ferromagnetic particles may be incorporated into a polymer matrix. Then, the particles are magnetized. Additionally or alternatively, soft ferromagnetic particles, superparamagnetic particles or ferrimagnetic particles may be incorporated into a polymer matrix. The particles, with a diameter between 5 nm to 5 μm, preferably between 30 nm to 100 nm can be incorporated by emulsion, molding or prilling. The polymer matrix can be bioresorbable (PLLA, PLGA, PDO, PCL for example) or non-bioresorbable such as silicone, PDMS, polyurethane.

Furthermore, the magnetic head part 100 comprises an outer layer 103 which comprises tracking members (not visible) in the form of radiopaque particles. The magnetic head part 100 has a diameter in the range of 300 μm to 1.5 mm, preferably 400 μm to 800 μm.

FIG. 17b shows another embodiment of a magnetic head part 100 which comprises a substantially spherical magnetic material 106 with a hollow tube 107. The hollow tube may be used for transmission or suction of fluid, in particular to create a vacuum, and/or deliver a liquid, a gas, a therapeutic solution, to move a therapeutic tool in the front of the device and/or microrobot or to guide optical or electric cables through . The hollow tube 107 has a diameter between 70 μm to 200 μm, preferably 0.1 mm and spans substantially across a central region of the magnetic head part 100. It would be conceivable, however, to arrange a hollow tube at a position which is laterally displaced from the central portion. The hollow tube 106 may be at least partially curved and/or straight. The magnetic head portion 100 had a diameter of 1.1 mm.

FIG. 17c shows another embodiment of a magnetic head portion 100 configured as a Janus particle. Here, the Janus particle comprises on portion of magnetic material 106 and a portion comprising an active material 108.

Additionally or alternatively, the Janus particles can be made of two different magnetic materials. For example, FePt and Fe₂O₃. In such a configuration, the magnetic particles exhibit a hard ferromagnetic behavior (FePt) and ferrimagnetic behavior (Fe₂O₃). In another configuration, one side is made of magnetic material and the other side is made of non-magnetic material. Nonmagnetic material can be metals such as NiTi or polymers such as polyurethane. The non-magnetic material can be used to setup or activate the therapeutic tool of the microrobot. As example, the non-magnetic material can be made of NiTi which will change its shape under a stimuli such as an increase of temperature which could be induced with an electric current for example.

FIG. 17d shows yet another embodiment of a magnetic head portion 100 comprising a magnetic material 106 configured as an outer shell. The inner core 109 may be any other suitable material which may be magnetic or non-magnetic. For example, the outer layer can be made of Fe₃O₄ with a thickness of 200 μm and the inner core can be made of FePt with a diameter of 400 μm. Alternatively, the outer layer can be made of a mixture of Fe₂O₃ and FePt with a thickness of 300 μm and the inner core can be made of PDMS with a diameter of 400 μm. This design contributes to reduce the rigidity of the microrobot.

It will be understood that any of the features described in the context of the FIGS. 17a-17d may be used for any devices disclosed herein, in particular combined with any other features disclosed herein.

FIG. 18a shows an embodiment of a medical device 10 comprising a magnetic head portion 100 and a controlling line 13 attached thereto. Here, the controlling line 13 is attached to an attachment area 101 which comprises a cyanoacrylate adhesive that provides attachment between the magnetic head part 100 and the controlling line 13. Furthermore, a protective layer 111 configured as a layer of epoxy resin is arranged on the magnetic head part 100. Here, the protective layer 111 is continuously arranged on the entire surface of the magnetic head part 100 and the attachment area 110. The controlling line 13, which is attached to the attachment area 110, protrudes through the protective layer 111. Thus, the protective layer seals the magnetic head part 100 from fluids that may be present in the surrounding of the medical device 10 and reduces corrosion effects. It will be understood that the protective layer may, in some alternative embodiments, not be in contact with the controlling line 13 and only cover partially the attachment area 110 in a circumferential area (see FIGS. 18b and 18d), which would still provide a fluid seal of the magnetic head part 100.

FIG. 18b shows a different embodiment of a medical device 10 which is similar to the embodiment of FIG. 18a. The medical device 10 comprises a magnetic head part 100 which is attached to a controlling line 13 via an attachment area. Here, the magnetic head part 100 is partially coated with a protective layer 110 which is formed as a band and covers an interface 112 between the attachment area 110 and the magnetic head part 100. The configuration of the protective layer shown here may provide sufficient corrosion reduction, in particular over a typical treatment time frame, such as to provide secure attachment of the controlling line 13 to the magnetic head part 100. However, advantageously, less material is needed to provide the protective layer 111, which may be more economical, more environmentally friendly, and may reduce the overall size of the medical device 10. It will be understood that the protective layer 111 in the embodiment shown here does not provide a fluid seal of the entire magnetic head part 100 as a proximal portion 113 and a central portion of the magnetic head part 100 are not covered by the protective layer 111. However, the interface 112 is fluid-sealed by the protective layer 111. It would be conceivable to also cover, in some alternative embodiments, the central portion 114 and the proximal portion 113 with protective layer 111 such as to provide a fluid-seal of the entire magnetic head part 100.

This approach allows to have two different coatings: one for securing the attachment of the controlling line with the magnetic part and one for the functionalization of the head of the microrobot. For example, a therapeutic tool such as a tank for a drug or a hook can be attached to the uncovered magnetic surface with a resin layer.

Alternatively, the magnetic head could also be made of two parts which are closed together with the controlling line. With such design, the controlling line is embedded into the magnetic part. For example, one magnetic part has a female design and the other part has a male design. Both parts have an area for the controlling line. The controlling line is compressed between the two parts during the assembly.

FIG. 18c shows yet another embodiment of a medical device 10. Here, a magnetic head portion 100 is continuously coated with a protective layer 111 on its entire surface. An attachment area 110, which comprises a cyanoacrylate glue, is arranged on an outside surface on the protective layer 111. The controlling line 13 is attached to the magnetic head part 100 via the attachment area 110. Such a configuration may provide particularly secure attachment of the controlling line 13.

FIG. 18d shows yet another embodiment of a medical device 10. The embodiment shown here is similar to the embodiment shown in FIG. 18b. Here, the protective layer 111 is also formed as a band that covers an interface 112 between the attachment area 110 and the magnetic head part 100. However, the band is configured to cover more than 50% of the surface of the magnetic head part 100. The attachment area 110, which is formed by a hot melt adhesive, is not covered by the protective layer 111. A proximal area 113 of the magnetic head part 100 is not covered by the protective layer 111. Here, the controlling line 13 is attachable to the attachment area 110 by heating the hot melt adhesive and securing the controlling line 13 thereto.

It will be understood that the features of the embodiments of FIGS. 18a-18d may be freely combined. In particular, any magnetic head portion 100 may be attached or separately attachable to a controlling line 13. Similarly, any of the configurations of the protective layer shown in FIGS. 18a-18d may be used in combination with any other embodiment or feature shown here or further disclosed herein.

1.-40. (canceled)

41. A medical device, said medical device comprising:
a body part; and
a tail part attached to the body part, wherein a controlling line is attached to the device, and wherein a stiffness of the controlling line is not sufficient to move the medical device to a target location.

42. The medical device according to claim 41, wherein the medical device has at least one of:
i) a drive for actively moving the device in a direction; and
ii) a control member for changing the movement direction within a body by external effects.

43. The medical device according to claim 41, wherein the medical device has a positioning means to determine the position of the medical device in the body.

44. The medical device according to claim 41, wherein the controlling line comprises a transmission cable to transmit one of energy and data.

45. The medical device according to claim 41, wherein the controlling line comprises a material selected from a group of materials consisting of a metal, metal composites, polymers, carbon fibres, graphene, fabric, silk, protein fibres, aramid, and carbon nanotubes.

46. The medical device according to claim 41, wherein the controlling line has a smaller cross-section than the medical device.

47. The medical device according to claim 41, wherein the medical device comprises a material that enables detection by an imaging technique selected from the group consisting of IRM, scanner, echography, X-ray and fluoroscopy.

48. The medical device according to claim 41, wherein the controlling line has an outer diameter of 10 to 1000 μm.

49. The medical device according to claim 41, wherein the body part contains a magnetic part.

50. The medical device according to claim 41, wherein the body part contains at least one functional unit.

51. The medical device according claim 41, wherein the body part comprises a compartment configured to store and release a drug.

52. The medical device according to claim 41, wherein the body part contains a transmitter configured to send data from the medical device to a receiver.

53. The medical device according to claim 41, wherein the medical device has a size of 8-2000 μm.

54. The medical device according to claim 41, wherein the body part and tail part of medical device comprise a material selected from the group consisting of: metal, plastic, glass, mineral, ceramic, carbohydrate, nitinol, carbon, biomaterial, and a biodegradable material.

55. The medical device according to claim 41, wherein the controlling line is removably attached to the device.

56. The medical device according to claim 55, wherein the controlling line is selectively detachable from the device.

57. The medical device according to claim 41, comprising a first and a second portion of the medical device, wherein the first portion is attached to the controlling line, and wherein the second portion is removably attached to the first portion, wherein the first portion is selectively detachable from the second portion of the medical device.

58. The medical device according to claim 41, comprising exactly one line formed by the controlling line.

59. The medical device according to claim 41, wherein the controlling line is not able to transmit data or energy.

60. The medical device according to claim 41, wherein the controlling line can be bent into a curve with a curvature radius of less than 3 mm without substantial material stress.

61. The medical device according to claim 41, wherein the controlling line is comprises a radiopaque material.

62. The medical device according to claim 61, wherein the radiopaque material is arranged as a separate cable associated with and parallel to the controlling line and/or as a coating on the controlling line.

63. The medical device according to claim 41, wherein the controlling line comprises a hydrophilic surface.

64. The medical device according to claim 41, wherein the controlling line comprises a surface with anti-thrombogeneic properties.

65. The medical device according to claim 41, wherein the controlling line has a surface that is coated with a hydrogel.

66. The medical device according to claim 41, wherein the controlling line is attached to the medical device by at least one of a knot, a clip, a welded connection, an adhesive connection, a mix of materials, and a chemical bond.

67. The medical device according to claim 41, further comprising a hollow tube arranged in parallel with respect to the controlling line.

68. The medical device according to claim 41, further comprising a trigger wire adapted to trigger a function of the device.

69. A method for controlling a medical device comprising the steps of:
inserting the medical device, wherein the medical device includes a body part and a tail part;
navigating the medical device to a target site without pushing a controlling line; and
removing the medical device from the target site, by pulling the controlling line.

70. A system for controlling a medical device comprising a medical device according to claim 49 and a magnetic field generator wherein the medical device is guidable by a magnetic field generated by the magnetic field generator.

71. The system according to claim 70, further comprising a control adapted to control the velocity of the medical device.

72. The system according to claim 70, wherein the system further comprises a coupling element adapted to be coupled to the controlling line in order to connect the coupling element to the device to control its velocity.

73. The system according to claim 70, wherein the control is further adapted to pull in and/or release the controlling line at a controlled velocity.

74. The system according to claim 70, wherein the control comprises a mechanism to control the position of the medical device.

75. A method of controlling a device in a fluid stream, wherein the device comprises a controlling line attached to the device, wherein the velocity of the device is controlled via the controlling line.

76. The method according to claim 75, wherein the velocity is reduced when the device approaches a bifurcation.

77. The method according to claim 75, further comprising a control which automatically controls the velocity of the device.

78. The method according to claim 77, wherein the control automatically detects bifurcations.

79. A medical device, comprising a magnetic head portion attached or attachable to a controlling line, wherein the medical device further comprises a protective layer wherein the protective layer is configured to provide a fluid seal at least of an area of the magnetic head portion attached or attachable to the connecting line.

80. A method of producing a medical device comprising the steps of:
providing a magnetic head portion with an attachment area attached or attachable to a controlling line; and
providing a protective layer at least partially covering and/or forming an interfacial area between the magnetic head portion and the attachment area.
